(19) United States
(12) Patent Application Publication
(10) Pub. No.: US 2026/0283919 A1
(43) Pub. Date: Sep. 24, 2026

(54) Aqueous Cosmetic Composition Comprising a Fatty Acid Monoester, a Neutralized Anionic Surfactant, a VP/Eicosene Copolymer, a Semicrystalline Polymer, a Latex, a Filler and a Polyol

(71) Applicant: L'OREAL, PARIS (FR)

(72) Inventors: XAVIER JALENQUES, CHEVILLY LARUE (FR); FRANCINE BALDO, CHEVILLY LARUE (FR); CAMILLE MOYNE, CHEVILLY LARUE (FR); JULIEN GOYENVALLE, CHEVILLY LARUE (FR)

(73) Assignee: L'OREAL, Paris (FR)

(21) Appl. No.: 19/484,542

(22) PCT Filed: Apr. 24, 2024
(86) PCT No.: PCT/EP2024/061274

§ 371 Date: Nov. 14, 2025

(30) Foreign Application Priority Data
May 15, 2023 (FR) FR2304809

(51) Int. Cl. A61K 8/37; A61K 8/19; A61K 8/26; A61K 8/34; A61K 8/36; A61K 8/41; A61K 8/81; A61Q 1/10

(52) U.S. Cl. CPC A61K 8/37; A61K 8/19; A61K 8/26; A61K 8/34; A61K 8/342; A61K 8/345; A61K 8/361; A61K 8/41; A61K 8/8152; A61K 8/8176; A61K 8/8182; A61Q 1/10; A61K 2800/43; A61K 2800/591; A61K 2800/594; A61K 2800/654; A61K 2800/87

(57) ABSTRACT

The present invention relates to a composition comprising, notably in a physiologically acceptable medium: (1) at least one linear fatty acid monoester of formula (I) below: R¹—O—R₂ (I) in which R₁ and R₂ are linear and saturated and have, independently of each other, a number of carbon atoms greater than or equal to 20, with Representing an acyl radical, and Representing an alkyl radical, and (2) at least one anionic surfactant, and (3) at least one base that is capable of partially or totally neutralizing said anionic surfactant, and (4) at least one vinylpyrrolidone/eicosene copolymer, and (5) at least one semicrystalline polymer, and (6) an aqueous phase, and (7) at least one film-forming polymer in the form of solid particles in suspension in the aqueous phase; and (8) at least one filler having a water absorption capacity <200 mL/100 g measured at 25° C. by the Wet Point method; and (9) at least one water-soluble liquid polyol.

### TECHNICAL FIELD

The present invention relates to the field of caring for and/or making up keratin materials, and is directed toward proposing compositions more particularly intended for making up the eyelashes or the eyebrows.

In general, compositions intended for making up keratin fibers, for example the eyelashes, aim to densify the thickness and the visual perception of the eyelashes and ultimately the look. These mascaras are described as aqueous or cream mascaras when they are formulated in an aqueous base, and anhydrous mascaras when they are formulated as a dispersion in an organic solvent medium.

A great diversity of cosmetic effects may be afforded by applying a mascara to keratin fibers and notably the eyelashes, for instance a volumizing, lengthening, thickening and more particularly charging makeup effect.

These effects are mostly adjusted through the amount and nature of the particles and most particularly those of the waxes present in the mascaras. In general, mascaras in fact have a significant amount of wax(es) and notably from 10% to 35% by weight of waxes, more generally from 15% to 30% by weight, relative to the total weight thereof.

For obvious reasons, improving the textures of mascara which condition the manifestation of one or more makeup effects is a constant preoccupation of cosmetic formulators.

Moreover, the specific effects associated with a particular formulation, for example charging and moreover providing excellent separation of the made-up eyelashes, are expected to be reproduced virtually identically by all the production batches of one and the same formulation.

In order to meet these expectations and/or objectives, it is therefore necessary to be capable of precisely adjusting the texture of a mascara and of reproducing it as faithfully as possible with batches that are not necessarily produced at the same time but which are identical in terms of ingredients and must therefore provide makeup effects that are in theory also identical.

### PRIOR ART

However, as specified above, most of the mascaras currently available are formulated with a significant amount of waxes. In point of fact, as detailed in Ullmann's Encyclopedia of Industrial Chemistry 2015, Wiley-VCH Verlag GmbH & Co. KGaA, Weinheim 10.1002/14356007.a28.pub2, most waxes do not consist of a single chemical compound, but are instead complex mixtures. They may be mixtures of oligomers and/or of polymers which, in many cases, also have varied molar masses, varied molar mass distributions and also varied degrees of branching. Thus, a polar wax is conventionally made up of a mixture of alkanes, fatty alcohols and fatty esters, the fatty-chain length of which varies according to the melting point.

It is therefore very difficult for the producers of these waxes to guarantee a rigorously identical composition for all production batches. More specifically, there may be, between several production batches of one and the same wax, a variability in terms of the chemical nature of some of its constituent compounds. Similarly, the proportionality of some of its constituent compounds may also vary between production batches.

For obvious reasons, these variabilities have a not insignificant impact on the properties of the wax and therefore on those of the mascara incorporating this wax in significant amount. Thus, two mascara formulations of identical composition and therefore produced from one and the same conventional wax and in the same amount, can nevertheless differ in terms of rheological properties and therefore of texture and mechanical properties of the deposition of the product on keratin fibers, such as resistance to flaking (presence of grains also known as flakes), if they were produced from two separate production batches of this wax.

Consequently, the use of conventional waxes, in particular in significant amount, in mascara compositions does not make it possible to guarantee for users the reproduction of the finely adjusted and totally identical rheological properties and mechanical properties of the deposition of the product on keratin fibers, such as resistance to flaking, in all the mascara specimens of one and the same composition.

In patent applications FR3080034, EP3781118, EP3897859, EP3897558 and EP3897519, aqueous compositions, notably mascaras, were proposed comprising at least one fatty acid monoester such as behenyl behenate, at least one fatty acid containing from 14 carbon atoms to less than 20 carbon atoms, at least one base that is capable of at least partially neutralizing said fatty acid, at least one base that is capable of at least partially neutralizing said fatty acid, and pigments, notably iron oxides. However, these compositions are not entirely satisfactory in terms of the stability of the evolution of the consistency over time, notably over several days and according to temperature variations.

Aqueous mascaras comprising behenyl behenate, a fatty acid, a neutralizing base, a semicrystalline polymer, a fatty alcohol and a nonvolatile hydrocarbon-based oil are known in patent FR3090334.

Thermosetting mascaras with a semicrystalline polymer and a sulfopolyester resin were proposed in patent FR2853528.

Patent application US 2015/174056 proposed cosmetic compositions for coating keratin fibers, of the emulsion type, including an aqueous phase, at least one wax, an emulsifying system, at least one film-forming polymer present in a dry matter content of greater than or equal to 5% by weight relative to the total weight of the composition, at least one organic, preferably polymeric, lipophilic gelling agent, in which the wax(es) and the emulsifying system are present in a respective total content such that the weight ratio of the wax(es) to the emulsifying system is less than or equal to 1. Example 2 and the comparative are compositions comprising 5.5% of conventional waxes, a VP/eicosene copolymer, a pigment, and stearic acid neutralized with triethanolamine.

Aqueous mascaras are known on the market
the commercial product Be Bold Colar Mascara (Mintel ID 5676593) containing behenyl behenate, neutralized stearic acid, waxes and black iron oxides;
the commercial product SuperDrama Volume and Lengthening Waterproof Mascara from Avon (Mintel ID 20600879) containing behenyl behenate, neutralized stearic acid, waxes and black iron oxides;
the commercial product Stunning Extra Lush Mascara from Ying Te LI Cosmetics (Mintel ID 8372591) containing behenyl behenate, potassium cetyl phosphate neutralized with a base, waxes, black iron oxides and vinylpyrrolidone/eicosene copolymer.

Aqueous compositions based on semicrystalline polymer, an silicone elastomer gel and a hydrophilic gelling agent of the ammonium polyacryloyldimethyl taurate type were proposed in patent U.S. Pat. No. 8,932,573. Examples describe mascaras with vinylpyrrolidone/eicosene copolymer, stearic acid, large amounts of conventional waxes and iron oxides.

Thus, a first aim of the present invention is to provide mascara compositions whose deposit on keratin materials such as eyelashes is, firstly, smoother, resulting in the absence of grains after application of the product, and secondly, with a longer product application time, denoted by the term “play time”. The application time is particularly important, notably as regards the comfort of use. In particular, a long application time allows a user to take more time to apply her make up without having to reapply the product several times on the keratin material.

A second aim of the present invention is to afford access to mascara compositions whose consistency is more stable over time, notably over several days and according to temperature variations.

A third aim of the present invention is to afford access to mascara compositions whose texturing properties are finely adjustable and reproducible.

A fourth aim of the present invention is to afford access to mascara compositions for which the mechanical properties of the deposit of the product on keratin fibers, such as the resistance to flaking, are finely adjustable and reproducible.

Another aim of the present invention is to provide a mascara architecture produced using a predominant weight proportion of single-component ingredients. The predominant use of single-component ingredients advantageously makes it possible to eliminate the risk of composition variability that may exist between several production batches of a multicomponent ingredient and therefore to eliminate its impact on the final properties of the mascara.

Another aim of the present invention is to provide a mascara architecture which makes it possible to significantly reduce or even dispense with the use of waxes, but which nevertheless remains very satisfactory in terms of makeup effect.

In the course of their research, the inventors have discovered, unexpectedly, that the aims as defined previously are achieved by using a composition comprising, notably in a physiologically acceptable medium:
at least one linear fatty acid monoester of formula (I) below:

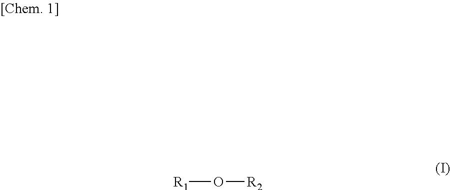

in which R₁ and R₂ are linear and saturated and have, independently of each other, a number of carbon atoms greater than or equal to 20, with R₁ representing an acyl radical, and R₂ representing an alkyl radical, and
at least one anionic surfactant, and
at least one base that is capable of partially or totally neutralizing said anionic surfactant, and
at least one vinylpyrrolidone/eicosene copolymer, and
at least one semicrystalline polymer, and
an aqueous phase, and
at least one film-forming polymer in the form of solid particles in suspension in the aqueous phase; and
at least one filler with a water absorption capacity <200 mL/100 g measured at 25° C. by the Wet Point method; and
at least one water-soluble liquid polyol.

In an unexpected manner, the inventors have in fact found that the formulation comprising at least one linear fatty acid monoester (1), at least one anionic surfactant (2) in a form partially or totally neutralized with a base (3), at least one vinylpyrrolidone/eicosene copolymer (4), at least one semicrystalline polymer (5), an aqueous phase (6), at least one film-forming polymer (7) in the form of solid particles suspended in the aqueous phase (6), at least one filler (8) with a water absorption capacity <200 mL/100 g, and at least one polyol (9), allows a smoother deposit to be produced after application to keratin materials such as eyelashes, resulting in the absence of grains and a longer product application time (play time) on said keratin materials.

Furthermore, the consistency and mechanical properties of the deposit of the formulation on the keratin fibers can be finely adjusted and guaranteed in terms of reproducibility.

As emerges from what follows, these new compositions are advantageous in several respects.

First of all, compounds (1) to (9) required according to the invention are as an individualized compound, a single-component compound or a compound with a well-defined number of components, as opposed to the majority of conventional waxes which are often multicomponent or even have an indefinite number of compounds, such as natural waxes and some synthetic waxes.

These two specificities are particularly advantageous since they make it possible to eliminate a risk of variability with regard to their respective compositions.

As emerges from the examples below, the compositions in accordance with the invention and based on the use of compounds (1) as texturing agent prove to be very satisfactory in terms of makeup effects.

Thus, compositions according to the invention may have a creamy texture which proves to be finely adjustable by virtue of the use of the required combination according to the invention.

The obtention of these properties is conditioned by the use of the compounds (1) to (9) and advantageously does not therefore require the additional presence of waxes, notably in significant amount.

Thus, the compositions according to the invention advantageously comprise less than 5% of waxes as defined below.

The term “waxes” refers to lipophilic compounds, which are solid at room temperature (25° C.) and at atmospheric pressure (760 mmHg), with a reversible solid/liquid change of state, which have a melting point of greater than or equal to 40° C., which may be up to 120° C.

For the purposes of the invention, the waxes to which this abovementioned amount limitation relates are distinct from those that may be embodied by the component which is a monoester of fatty acid(s) (1) required according to the invention and additives of the fatty alcohol type.

This discovery forms the basis of the invention.

### SUBJECTS OF THE INVENTION

Thus, according to one of its aspects, the present invention relates to a composition comprising, notably in a physiologically acceptable medium:
at least one linear fatty acid monoester of formula (I) below:

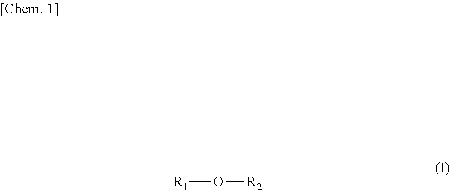

in which R₁ and R₂ are linear and saturated and have, independently of each other, a number of carbon atoms greater than or equal to 20, with R₁ representing an acyl radical, and R₂ representing an alkyl radical, and
at least one anionic surfactant, and
at least one base that is capable of partially or totally neutralizing said anionic surfactant, and
at least one vinylpyrrolidone/eicosene copolymer, and
at least one semicrystalline polymer, and
an aqueous phase, and
at least one film-forming polymer in the form of solid particles in suspension in the aqueous phase; and
at least one filler having a water absorption capacity <200 mL/100 g measured at 25° C. by the Wet Point method; and
at least one water-soluble liquid polyol.

A second subject of the present invention is a cosmetic process for making up and/or caring for human keratin materials, such as the skin, notably the area around the eyes, the area around the eyelashes, the area around the eyebrows; keratin fibers such as the eyelashes and the eyebrows, which consists in applying to said keratin materials a composition as defined previously.

### DEFINITIONS

In the context of the present invention, the term “keratin material” notably means the skin, notably the skin around the eyelashes, the skin around the eyebrows; keratin fibers such as the eyelashes and the eyebrows. For the purposes of the present invention, this term “keratin fibers” also extends to synthetic false eyelashes.

The term “physiologically acceptable” means compatible with the skin and/or its integuments, which has a pleasant color, odor and feel, and which does not cause any unacceptable discomfort (stinging or tautness) liable to discourage the consumer from using this composition.

According to another of its aspects, the present invention relates to a process, notably a cosmetic process, for caring for and/or making up keratin materials, in particular the skin around the eyelashes, the skin around the eyebrows, the eyelashes and/or the eyebrows, comprising at least one step which consists in applying to said keratin materials, in particular the eyelashes and/or the eyebrows.

The term “particulate matter” means any compound in the form of particles which are insoluble and dispersible in the composition of the invention.

The term “filler” should be understood as meaning a colorless or white solid particle of any form, which is in an insoluble form dispersed in the medium of the composition.

### Fatty Acid Monoester

A composition according to the invention comprises at least one linear fatty acid monoester.

A composition according to the invention may comprise at least 5.0% by weight, preferably at least 6.0% by weight, better still at least 7.0% by weight of linear fatty acid monoester(s), relative to the total weight of the composition.

According to a particularly preferred embodiment of the invention, the fatty acid monoester(s) are present in the composition in a content ranging from 6.0% to 35.0% by weight, preferably from 7.0% to 30.0% in weight, or even preferably from 8.0% to 28.0% by weight, relative to the total weight of the composition.

The linear fatty acid monoester(s) (1) under consideration according to the invention correspond to formula (I) below:

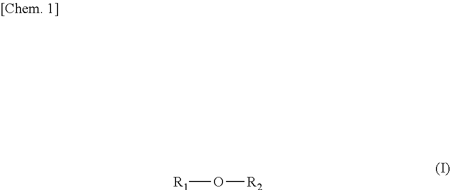

in which R₁ and R₂ are linear and saturated and have, independently of each other, a number of carbon atoms greater than or equal to 20, with R₁ representing an acyl radical, and R₂ representing an alkyl radical.

This or these fatty acid monoester(s) are used during the preparation of a composition according to the invention, in an individualized form or in the form of a mixture exclusively comprising linear fatty acid monoesters of formula (I).

In a preferred embodiment, the fatty acid monoester(s) have a melting point greater than 50° C.

The melting point may be measured by any known method and in particular using a differential scanning calorimeter (DSC).

According to a preferred embodiment of the invention, the acyl and alkyl radicals representing R₁ and R₂, respectively, are chosen in such a way that compound (I) is solid at a temperature of less than or equal to 30° C.

According to a particularly preferred embodiment of the invention, R₁ and R₂ are, respectively, acyl and alkyl radicals having a number of carbon atoms ranging from 20 to 30, preferably from 20 to 24.

According to a particularly preferred embodiment, R₁ and R₂ are, respectively, acyl and alkyl radicals having the same number of carbon atoms.

In particular, the fatty acid monoester according to the invention is chosen from arachidyl arachidate and behenyl behenate.

According to a particularly preferred embodiment of the invention, the linear fatty acid monoester is a behenyl behenate.

A behenyl behenate that is suitable for use in the composition according to the invention may notably be Kester Wax K-72® sold by the company Koster Keunen, Dub BB® sold by Stéarinerie Dubois, or Dermowax BB® sold by Alzo.

### Anionic Surfactant

As specified above, the fatty acid monoester(s) (1) used according to the invention are combined with at least one anionic surfactant that is partially or totally neutralized with a base (3).

For the purposes of the present invention, the term “surfactant” means an amphiphilic chemical compound, that is to say a compound having in its structure two parts of different polarity. Generally, one is lipophilic (soluble or dispersible in an oily phase). The other is hydrophilic (soluble or dispersible in water). Surfactants are characterized by the value of their HLB (hydrophilic-lipophilic balance), the HLB being the ratio of the hydrophilic part to the lipophilic part in the molecule. The term “HLB” is well known to those skilled in the art and is described, for example, in “The HLB System. A Time-Saving Guide to Emulsifier Selection” (published by ICI Americas Inc.; 1984).

According to a preferential form of the invention, the anionic surfactants in accordance with the invention have an HLB of greater than or equal to 8. The HLB of the anionic surfactant(s) used according to the invention can be determined by the Griffin method.

The term “anionic surfactant” means any negatively charged amphiphilic molecule.

According to a preferred embodiment of the invention, the anionic surfactant is present in a content ranging from 3.5% to 20.0% by weight, more preferentially from 4.0% to 20.0% by weight, better still from 4.5% to 15.0% by weight, even better still from 5.0% to 15.0% by weight relative to the total weight of the composition.

The anionic surfactant(s) in accordance with the invention are preferably chosen from:
i. C12-C20 monoalkyl phosphates;
ii. alkyl sulfates, and in particular alkyl ether sulfates, alkylamido ether sulfates, alkylaryl polyether sulfates, monoglyceride sulfates;
iii. alkylsulfonates, alkylamidesulfonates, alkylarylsulfonates, α-olefinsulfonates, paraffinsulfonates;
iv. alkyl sulfosuccinates, alkyl ether sulfosuccinates, alkylamide sulfosuccinates; alkyl sulfosuccinamates;
v. acyl sarcosinates, acyl glutamates, acyl isethionates, N-acyl taurates and acyl lactylates;
vi. fatty acids containing from 14 to less than 20 carbon atoms;
vii mixtures thereof.

According to a particular form of the invention, said anionic surfactant(s) are chosen from:
C12-C20 monoalkyl phosphates;
fatty acids containing from 14 to less than 20 carbon atoms;
mixtures thereof.

Use will be made more particularly of fatty acids containing from 14 to less than 20 carbon atoms.

### a) C12-C20 Monoalkyl Phosphates

The designation “monoalkyl” means that the phosphate element is linked to a single C12-C20 alkyl chain, unless otherwise specified. The monoalkyl phosphate(s) (including phosphine oxide(s)) that may be used in the compositions according to the present patent application are chosen from C14-C20 and preferably C16-C18 monoalkyl phosphates and mixtures thereof.

Preferably, they are chosen from monocetyl phosphate, monostearyl phosphate and monocetearyl phosphate.

In particular, use will be made of the anionic surfactant in its form neutralized with potassium hydroxide (KOH), namely the potassium salt of monocetyl phosphate having the INCI name Potassium Cetyl Phosphate, for example sold under the names Amphisol K® (DSM Nutritional Products), Amphisol A® (DSM Nutritional Products), Arlatone MAP® (Uniqema), Crodafos MCA® (Croda).

b) Fatty Acid Containing from 14 to Less than 20 Carbon Atoms

The fatty acid according to the invention comprises from 14 to less than 20 carbon atoms. According to a preferred embodiment of the invention, the fatty acid comprises from 16 to less than 20 carbon atoms. According to a particularly preferred embodiment, the number of carbon atoms ranges from 16 to 18.

In particular, the fatty acid(s) according to the invention are chosen from linear fatty acids, saturated fatty acids and mixtures thereof.

According to a particularly advantageous embodiment of the invention, the fatty acid of the ionic surfactant is linear and saturated.

According to a particular embodiment of the invention, the fatty acid(s) are chosen from palmitic acid, stearic acid and mixtures thereof, and preferably comprise at least stearic acid having the INCI name Stearic Acid.

Thus, according to another embodiment of the invention, the composition uses, as fatty acid (2), a mixture of C16-C18 fatty acids, preferably a mixture of fatty acids containing 16 carbon atoms, such as palmitic acid, and of fatty acids containing 18 carbon atoms, such as stearic acid.

A preferred stearic acid that is suitable for use in the invention is, for example, Stearic Acid 1850® sold by the company Southern Acids or the product Stearine TP 1200 Pastilles® (DUB 50P®) by the company Stéarinerie Dubois.

### Base

The composition according to the invention comprises at least one base (3). This base may be organic or inorganic.

According to a first variant, the base (3) is at least one organic base.

Preferably, the base of organic origin is chosen from amino acids such as arginine; alkanolamines such as monoethanolamine, diethanolamine, triethanolamine, triisopropanolamine, aminomethylpropanol; primary (poly) hydroxyalkylamines such as 2-amino-2-(hydroxymethyl) propane-1,3-diol (also known as tromethamine) and aminomethylpropanediol; and mixtures thereof.

According to a particular embodiment, the base is a primary (poly)hydroxyalkylamine.

According to another particular embodiment, the base is an amino acid, in particular arginine.

The term “primary (poly)hydroxyalkylamine” in particular means a primary dihydroxyalkylamine, it being understood that the term “primary” means a primary amine function, i.e. —NH₂, the alkyl group being a linear or branched C1-C8 and preferably a branched C4 hydrocarbon-based chain, such as 1,3-dihydroxy-2-methylpropyl. The primary (poly) hydroxyalkylamine is preferentially 1,3-dihydroxy-2-methyl-2-propylamine (also known as aminomethylpropanediol or AMPD).

According to a preferred embodiment of the invention, the base of organic origin is aminomethylpropanediol.

Such an aminomethylpropanediol that is suitable for use in the invention is, for example, AMPD Ultra PC® sold by the company Angus (Dow Corning).

According to a second variant, the base is at least one inorganic base.

This inorganic base is chosen from ammonium (NH₄⁺) and alkali metal hydroxides.

Preferably, the inorganic base is chosen from sodium hydroxide and potassium hydroxide, and mixtures thereof.

The amount of base is adjusted so as to obtain sufficient neutralization to confer effective ionicity on the associated fatty acid (2).

Preferably, the base (3) is present in an amount sufficient to neutralize some or all of the carboxylic functions of the fatty acid(s) (2) comprising from 14 to less than 20 carbon atoms.

For example, the composition according to the invention may comprise at least 0.1% by weight, better still at least 0.15% by weight, of base(s) (3), relative to the total weight of the composition.

According to a preferred embodiment of the invention, the base (3) is present in a content ranging from 0.2% to 3.0% by weight, preferably from 0.3% to 2.0% by weight of base(s), in particular of sodium hydroxide, potassium hydroxide or aminomethylpropanediol, relative to the total weight of the composition according to the invention.

The anionic surfactant (2) and the base (3) making up the neutralized ionic surfactant according to the invention may be introduced into the composition in the form of one and the same commercial material, or one after the other in the form of two distinct commercial materials.

Preferably, the anionic surfactant (2) and the base (3) will be introduced into the composition in the form of two distinct commercial materials.

According to a particular embodiment of the invention, the compositions according to the invention contain at least the stearic acid anionic surfactant having the INCI name Stearic acid neutralized with aminomethylpropanediol.

According to another particular mode of the invention, the compositions according to the invention contain at least monocetyl phosphate anionic surfactant in its form totally neutralized with potassium hydroxide (KOH), namely the potassium salt of monocetyl phosphate, having the INCI name Potassium Cetyl Phosphate.

According to another particular mode of the invention, the compositions according to the invention contain at least one mixture of monocetyl phosphate potassium salt, having the INCI name Potassium cetyl phosphate and stearic acid neutralized with aminomethylpropanediol.

### Vinylpyrrolidone/Eicosene Copolymer

The composition according to the invention comprises at least one copolymer of vinylpyrrolidone and eicosene (C20 olefin) (4) of the following structure:

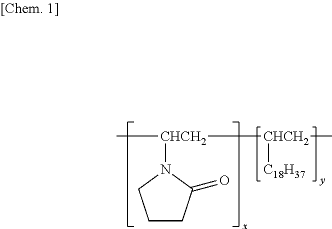

and having the INCI name: VP/Eicosene Copolymer, such as the commercial products sold under the names Antaron V-220® and Ganex V-220® by the company Ashland Inc. and Unimer U-15® by the company Givaudan Active Beauty.

The composition according to the invention comprises at least one copolymer of vinylpyrrolidone and eicosene, preferably in a concentration ranging from 0.5% to 10% by weight relative to the total weight of the composition, preferably ranging from 1% to 5% by weight relative to the total weight of the composition.

### Semicrystalline Polymer

A composition according to the invention comprises at least one semicrystalline polymer (5).

The composition according to the invention preferably comprises at least 2.0% by weight, more preferentially from 3.0% to 20.0% by weight, better still from 4.0% to 15.0% by weight, even better still from 5.0% to 15.0% by weight of semicrystalline polymer(s) (5), relative to the total weight of the composition.

For the purposes of the invention, the term “polymers” means compounds including at least two repeating units, preferably at least three repeating units and more especially at least ten repeating units.

For the purposes of the invention, the term “semicrystalline polymer” means polymers including a crystallizable portion and an amorphous portion and having a first-order reversible change of phase temperature, in particular of melting (solid-liquid transition). The crystallizable portion is preferably a chain that is lateral (or a chain that is pendent) relative to the backbone.

Besides the crystallizable chains or blocks, the blocks of the polymers are amorphous.

For the purposes of the invention, the term “crystallizable chain or block” means a chain or block which, if it were alone, would change from the amorphous state to the crystalline state reversibly, depending on whether the temperature is above or below the melting point. For the purposes of the invention, a chain is a group of atoms, which is pendent or lateral relative to the polymer backbone.

When the crystallizable portion is a chain that is pendent relative to the backbone, the semicrystalline polymer may be a homopolymer or a copolymer.

Preferably, the semicrystalline polymer (5) has an organic structure.

The term “organic compound” or “having an organic structure” refers to compounds containing carbon atoms and hydrogen atoms and optionally heteroatoms such as S, O, N or P, alone or in combination.

The semicrystalline polymer(s) according to the invention are solids at room temperature (25° C.) and atmospheric pressure (760 mmHg), the melting point of which is greater than or equal to 30° C.

The melting point values correspond to the melting point measured using a differential scanning calorimeter (D.S.C.) such as the calorimeter sold under the name DSC 30 by the company Mettler, with a temperature rise of 5° C. or 10° C. per minute (the melting point considered is the point corresponding to the temperature of the most endothermic peak in the thermogram).

According to a particular embodiment, the semicrystalline polymer(s) (5) used in the composition of the invention have a melting point m.p. of less than 95° C., preferably less than 85° C. The semicrystalline polymer(s) may thus have a melting point m.p. ranging from 30 to 95° C. and preferably from 40 to 85° C. This melting point is preferably a first-order change of state temperature.

According to the invention, the semicrystalline polymers (5) are advantageously soluble in the fatty phase, notably to at least 1.0% by weight, at a temperature above their melting point. Preferably, the polymer backbone of the semicrystalline polymers is soluble in the fatty phase at a temperature above their melting point.

Preferably, the crystallizable blocks or chains of the semicrystalline polymers (5) represent at least 30% of the total weight of each polymer and better still at least 40%.

When the semicrystalline polymers (5) of the invention are crystallizable block polymers, they may be block or multiblock copolymers. They may be obtained by polymerizing a monomer bearing reactive (or ethylenic) double bonds or by polycondensation. When the polymers of the invention are polymers bearing crystallizable side chains, these side chains are advantageously in random or statistical form.

Preferably, the semicrystalline polymers (5) of the invention are of synthetic origin.

According to a preferred embodiment, the semicrystalline polymer (5) is chosen from homopolymers and copolymers including units resulting from the polymerization of one or more monomers bearing crystallizable hydrophobic side chain(s).

The semicrystalline polymers (5) that may be used in the invention may be chosen in particular from homopolymers or copolymers, in particular those bearing at least one crystallizable side chain, such as those described in U.S. Pat. No. 5,156,911.

In a preferred embodiment, the crystallizable side chain(s) are hydrophobic.

These homopolymers or copolymers may result:
from the polymerization, notably radical polymerization, of one or more monomers bearing double bond(s) that are reactive or ethylenic with respect to a polymerization, namely bearing a vinyl, (meth)acrylic or allylic group,
from the polycondensation of one or more monomers bearing co-reactive groups (carboxylic acid, sulfonic acid, alcohol, amine or isocyanate), for instance polyesters, polyurethanes, polyethers or polyureas.

In general, the crystallizable units (chains or blocks) of the semicrystalline polymers (5) according to the invention are derived from monomer(s) containing crystallizable block(s) or chain(s), used for manufacturing semicrystalline polymers. These polymers are preferably chosen notably from homopolymers and copolymers resulting from the polymerization of at least one monomer containing crystallizable chain(s) that may be represented by the formula below:

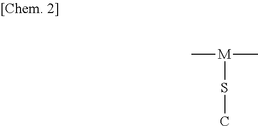

with M representing an atom of the polymer backbone, C representing a crystallizable group, and S representing a spacer, the crystallizable “—S—C” chains being hydrocarbon-based aliphatic or aromatic chains, including saturated or unsaturated hydrocarbon-based alkyl chains, which are for example C10-C40, preferably C10-C30. “C” notably represents a linear or branched or cyclic group (CH₂)ₙ, with n being an integer ranging from 10 to 40. Preferably, “C” is a linear group. Preferably, “S” and “C” are different.

When the crystallizable chains are hydrocarbon-based aliphatic chains, they include hydrocarbon-based alkyl chains containing at least 10 carbon atoms and not more than 40 carbon atoms and better still not more than 30 carbon atoms. They are notably aliphatic chains or alkyl chains containing at least 10 carbon atoms, and they are preferably C10-C40, preferably C10-C30 alkyl chains.

Preferably, the crystallizable chains are C10-C30 hydrocarbon-based aliphatic chains.

As examples of semicrystalline homopolymers or copolymers containing crystallizable chain(s) that are suitable for use in the invention, mention may be made of those resulting from the polymerization of one or more of the following monomers: saturated alkyl (meth)acrylates with the alkyl group being C10-C30, N-alkyl (meth)acrylamides with the alkyl group being C10 to C30, vinyl esters containing alkyl chains with the alkyl group being C10 to C30, vinyl ethers containing alkyl chains with the alkyl group being C10 to C30, C10 to C30 alpha-olefins, for instance octadecene, para-alkylstyrenes with an alkyl group including from 10 to 30 carbon atoms, and mixtures thereof.

When the polymers result from a polycondensation, the hydrocarbon-based crystallizable chains as defined above are borne by a monomer that may be a diacid, a diol, a diamine or a diisocyanate.

When the polymers that are subjects of the invention are copolymers, they additionally contain from 0% to 50% of groups Y which is a polar monomer, a nonpolar monomer or a mixture of the two.

When Y is a polar monomer, it is either a monomer bearing polyoxyalkylene groups (notably oxyethylene and/or oxypropylene groups), a hydroxyalkyl (meth)acrylate, for instance hydroxyethyl acrylate, (meth)acrylamide, an N-alkyl (meth)acrylamide, an N,N-dialkyl (meth)acrylamide, for instance N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.

When Y is a nonpolar monomer, it may be an ester of the linear, branched or cyclic alkyl (meth)acrylate type, a vinyl ester, an alkyl vinyl ether, an alpha-olefin, styrene or styrene substituted with a C1 to C10 alkyl group, for instance α-methylstyrene, or a macromonomer of the polyorganosiloxane type containing vinyl unsaturation.

Preferably, the semicrystalline polymers containing crystallizable side chain(s) are alkyl (meth)acrylate or alkyl (meth)acrylamide homopolymers with an alkyl group as defined above, and notably of C10-C30, copolymers of these monomers with a hydrophilic monomer preferably of different nature from (meth)acrylic acid, for instance N-vinylpyrrolidone or hydroxyethyl (meth)acrylate, and mixtures thereof.

It is also possible to use the semicrystalline polymers obtained by copolymerization of behenyl acrylate and of acrylic acid or of N-vinylpyrrolidone, as described in U.S. Pat. No. 5,519,063.

Advantageously, the semicrystalline polymer(s) containing one or more crystallizable side chain(s) have a weight-average molecular mass Mw ranging from 5000 g/mol to 1 000 000 g/mol, preferably from 10 000 g/mol to 800 000 g/mol, preferentially from 15 000 g/mol to 500 000 g/mol, and more preferably from 80 000 g/mol to 200 000 g/mol.

According to a particular embodiment of the invention, the semicrystalline polymer may be chosen from homopolymers and copolymers resulting from the polymerization of at least one one monomer containing crystallizable side chain(s) chosen from saturated C10 to C30 alkyl (meth)acrylates, which may be represented by the following formula:

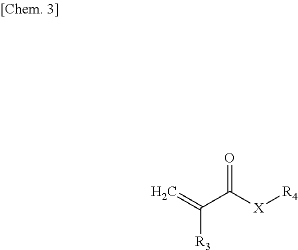

in which R₃ is H or CH₃, R₄ represents a C10 to C30 alkyl group and X represents O.

According to a more particular embodiment of the invention, the semicrystalline polymer (5) results from the polymerization of monomers containing crystallizable side chain(s), chosen from saturated C10 to C30 alkyl (meth)acrylates.

The semicrystalline polymers (5) containing a crystallizable side chain may be chosen from copolymers resulting from the copolymerization of acrylic acid and C10 to C30 alkyl (meth)acrylate, notably such as those described in U.S. Pat. No. 5,156,911.

The semicrystalline polymers (5) may notably be those described in Examples 3, 4, 5, 7 and 9 of patent U.S. Pat. No. 5,156,911, and more particularly those obtained by the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of polyoctadecyl methacrylate in a 2.5/97.5 ratio.

As specific examples of the semicrystalline polymers (5) that may be used in the composition according to the invention, mention may be made of the Intelimer® products from the company Landec, described in the brochure “Intelimer® Polymers”, Landec IP22®. These polymers are in solid form at room temperature (25° C.). They bear crystallizable side chains. They are poly(C10-C30)alkyl acrylates, which are particularly suitable as semicrystalline polymers (5) that may be included in a composition in accordance with the present invention.

According to a particularly preferred embodiment of the invention, the semicrystalline polymer(s) (5) according to the invention are derived from a monomer containing a crystallizable chain chosen from saturated C10 to C30 alkyl (meth)acrylates and more particularly from poly(stearyl acrylate)s, poly(behenyl acrylate)s, and mixtures thereof.

Preferably, the semicrystalline polymers (5) that are suitable for use in the invention are those having the INCI name: Poly C10-30 Alkyl Acrylate, notably polystearyl acrylate, in particular the product sold under the name Tego SP 13-1 3® from the company Evonik Corporation, which is a polystearyl acrylate with a melting point of 49°±5° C., or polybehenyl acrylate, sold under the name Tego SP 13-6®, from the company Evonik Corporation, which is a polybehenyl acrylate with a melting point equal to 66°±5° C.

According to a particularly preferred embodiment, the semicrystalline polymer (5) is at least one poly(behenyl acrylate).

Examples of homopolymers or copolymers that are suitable as semicrystalline polymers (5) for the invention preferably include from 50% to 100% by weight of units resulting from the polymerization of one or more monomers bearing a crystallizable hydrophobic side chain.

### Film-Forming Polymer (7) in Aqueous Suspension

According to a particular form of the present invention, the composition comprises at least one film-forming polymer (7) in the form of solid particles in suspension in the aqueous phase (6) of the composition.

In the present patent application, the term “film-forming polymer” means a polymer that is capable of forming, by itself or in the presence of an auxiliary film-forming agent, a continuous deposit on a support, at a temperature of 20° C.

Said solid film-forming polymer particles (7) may be used either per se and are in suspension in the aqueous phase of the composition, or in the form of particles in aqueous dispersion (latex or pseudolatex).

Such a film-forming polymer present in the form of particles in aqueous dispersion is generally known as a (pseudo) latex, i.e. a latex or pseudolatex. Techniques for preparing these dispersions are well known to those skilled in the art.

The composition according to the invention may comprise one or more types of particle, these particles possibly varying as regards their size, their structure and/or their chemical nature.

The film-forming polymer(s) (7) may be present in a solids content ranging from 0.5% to 10% by weight, relative to the total weight of the composition, preferably ranging from 1% to 5% by weight, relative to the total weight of the composition.

These solid particles may be of anionic, cationic or neutral nature and may constitute a mixture of solid particles of different nature.

In the present invention, the term “aqueous” refers to a liquid medium based on water and/or hydrophilic solvents. This aqueous liquid medium may be constituted essentially of water. It may also comprise a mixture of water and of water-miscible organic solvent(s) (miscibility in water of greater than 50% by weight at 25° C.), such as lower monoalcohols containing from 2 to 5 carbon atoms, such as ethanol or isopropanol, glycols containing from 3 to 8 carbon atoms, such as propylene glycol, 1,3-butylene glycol or dipropylene glycol, C3-C4 ketones or C2-C4 aldehydes.

Among the film-forming polymers (7) that may be used in the composition of the present invention, mention may be made of synthetic polymers, of free-radical type or of polycondensate type, and polymers of natural origin, and mixtures thereof. In general, these polymers may be statistical polymers, block copolymers of A-B type, of A-B-A or ABCD multiblock type, or even grafted polymers.

### Free-Radical Film-Forming Polymer

The term “free-radical polymer” means a polymer obtained by polymerization of unsaturated and notably ethylenically unsaturated monomers, each monomer being capable of homopolymerizing (unlike polycondensates).

The film-forming polymers (7) of free-radical type may notably be acrylic and/or vinyl homopolymers or copolymers.

The vinyl film-forming polymers may result from the polymerization of ethylenically unsaturated monomers containing at least one acid group and/or esters of these acid monomers and/or amides of these acid monomers.

Ethylenically unsaturated monomers containing at least one acid group or monomer bearing an acid group that may be used include α,β-ethylenic unsaturated carboxylic acids such as acrylic acid, methacrylic acid, crotonic acid, maleic acid or itaconic acid. (Meth)acrylic acid and crotonic acid are used in particular, and more particularly (meth)acrylic acid.

The esters of acidic monomers are advantageously chosen from (meth)acrylic acid esters (also known as (meth)acrylates), notably (meth)acrylates of an alkyl, in particular of a C1-C20 and more particularly C1-C8 alkyl, (meth)acrylates of an aryl, in particular of a C6-C10 aryl, and (meth)acrylates of a hydroxyalkyl, in particular of a C2-C6 hydroxyalkyl.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters are in particular alkyl (meth)acrylates.

According to the present invention, the alkyl group of the esters may be fluorinated, or perfluorinated, i.e. some or all of the hydrogen atoms of the alkyl group are replaced with fluorine atoms.

Examples of amides of the acid monomers that may be mentioned are (meth)acrylamides, and notably N-alkyl(meth)acrylamides, in particular of a C2-C12 alkyl. Among the N-alkyl(meth)acrylamides that may be mentioned are N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

Mention may also be made of polymers resulting from free-radical polymerization of one or more free-radical monomers inside and/or partially at the surface of pre-existing particles of at least one polymer chosen from the group consisting of polyurethanes, polyureas, polyesters, polyesteramides and/or alkyds. These polymers are generally referred to as “hybrid polymers”.

### Polycondensate

As film-forming polymer (7) of polycondensate type, mention may be made of anionic, cationic, nonionic or amphoteric polyurethanes, acrylic polyurethanes, polyvinylpyrrolidone-polyurethanes, polyester-polyurethanes, polyether-polyurethanes, polyureas, polyurea/polyurethanes and silicone polyurethanes, and mixtures thereof.

The film-forming polyurethane may be, for example, an aliphatic, cycloaliphatic or aromatic polyurethane, polyurea/urethane or polyurea copolymer including, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block including fluoro groups.

The film-forming polyurethanes as defined in the invention may also be obtained from branched or unbranched polyesters or from alkyds including labile hydrogens, which are modified by reaction with a diisocyanate and a difunctional organic compound (for example dihydro, diamino or hydroxyamino), also including either a carboxylic acid or carboxylate group, or a sulfonic acid or sulfonate group, or alternatively a neutralizable tertiary amine group or a quaternary ammonium group.

Among the film-forming polycondensates, mention may also be made of polyesters, polyesteramides, fatty-chain polyesters, polyamides and epoxyester resins.

The polyesters may be obtained, in a known manner, by polycondensation of dicarboxylic acids with polyols, notably diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers can be used alone or as a combination of at least two dicarboxylic acid monomers. The choice is made in particular, among these monomers, of phthalic acid, isophthalic acid and terephthalic acid.

The diol may be chosen from aliphatic, alicyclic and aromatic diols. The diol used is chosen in particular from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner similar to that for the polyesters, by polycondensation of diacids with diamines or amino alcohols. Diamines that may be used are ethylenediamine, hexamethylenediamine and meta- or para-phenylenediamine. An amino alcohol that may be used is monoethanol-amine.

Use will be made, as a particular form of polycondensate film-forming polymer, of a polyester-polyurethane in aqueous dispersion having the INCI name: Polyurethane-35, such as the commercial product Baycusan C 1010/1®, and/or the product having the INCI name: Polyurethane-48, such as the commercial product Baycusan C 1008/1®, which are sold by the company Covestro.

### Polymer of Natural Origin

Use may be made in the present invention of optionally modified polymers of natural origin, such as shellac resin, sandarac gum, dammar resins, elemi gums, copal resins, water-insoluble cellulose-based polymers such as nitrocellulose, modified cellulose esters notably including carboxyalkyl cellulose esters such as those described in patent application US 2003/185 774, and mixtures thereof.

According to a particular embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylpropylmethacrylamidoammonium chloride terpolymer dispersions, dispersions of polyurethane/polyacrylic hybrid polymers and dispersions of particles of core-shell type, and mixtures thereof.

Various types of aqueous dispersions, notably commercial aqueous dispersions, which are suited to the preparation of the composition in accordance with the present invention are detailed below.
1/ Thus, according to a preferred embodiment of the invention, the aqueous dispersion of polymer particles is an aqueous dispersion of acrylic polymer.

The acrylic polymer may be a styrene/acrylate copolymer and notably a polymer chosen from copolymers resulting from the polymerization of at least one styrene monomer and at least one C1-C18 alkyl (meth)acrylate monomer. Mention may be made, as styrene monomer which can be used in the invention, for example, of styrene or α-methylstyrene, and in particular styrene.

The C1-C18 alkyl (meth)acrylate monomer is in particular a C1-C12 alkyl (meth)acrylate and more particularly a C1-C10 alkyl (meth)acrylate. The C1-C18 alkyl (meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

Use may be made according to the invention, as acrylic polymer in aqueous dispersion, of:
the aqueous dispersions of acrylic polymer which are sold under the names

Acronal DS-6250® by the company BASF, Neocryl A-45®, Neocryl XK-90®, Neocryl A-1070®, Neocryl A-1090®, Neocryl BT-62®, Neocryl A-1079® and Neocryl A-523® by the company DSM, Joncryl 95® and Joncryl 8211® by the company BASF, Daitosol 5000 AD®, Epitex 66 Polymer® in emulsion form by the company Dow Corning (INCI name: Acrylates Copolymer) or Daitosol 5000 SJ® (INCI name: Acrylates/Ethylhexyl Acrylate Copolymer) by the company Daito Kasei Kogyo; Syntran 5760 CG® (INCI name: Styrene/Acrylates/Ammonium Methacrylate Copolymer) by the company Interpolymer.
2/ According to one embodiment variant of the invention, the aqueous dispersion of polymer particles is an aqueous dispersion of polyester-polyurethane and/or polyether-polyurethane particles, in particular in anionic form.

The anionic nature of the polyester-polyurethanes and of the polyether-polyurethanes used according to the invention is due to the presence in their constituent units of groups bearing a carboxylic acid or sulfonic acid function.

The polyester-polyurethane or polyether-polyurethane particles used according to the invention are generally sold in aqueous dispersion form.

The particle content of said dispersions currently available on the market ranges from about 20% to about 50% by weight relative to the total weight of the dispersion.

Among the anionic polyester-polyurethane dispersions that may be used in the aqueous varnishes according to the invention, mention may be made in particular of the product sold under the name Avalure UR 405® by the company Noveon.

Among the anionic polyether-polyurethane particle dispersions that may be used according to the invention, mention may be made in particular of the products sold under the name Avalure UR 450® by the company Noveon and under the name Neorez R 970® by the company DSM, Baycusan C 1010® (INCI name: Polyurethane-35), such as the product sold under the name Baycusan C 1010/1®, and Baycusan C 1008/1® (INCI name: Polyurethane-48 (and) Methylpropanediol (and) Caprylyl Glycol (and) Phenylpropanol), which are sold by the company Covestro.

According to a particular embodiment of the invention, use may be made of a mixture of commercial dispersions consisting of anionic polyester-polyurethane particles as defined above and of anionic polyether-polyurethane particles also defined above.

For example, use may be made of a mixture consisting of the dispersion sold under the name Sancure 861® or a mixture of the product sold under the name Avalure UR 405® and of the product sold under the name Avalure UR 450®, these dispersions being sold by the company Noveon.

As other examples of film-forming polymers in accordance with the invention, mention may also be made of:
the polycondensates of polyamide type resulting from the condensation between (a) at least one acid chosen from the acids as described in U.S. Pat. Nos. 5,874,069, 5,919,441, 6,051,216 and 5,981,680, for instance those sold under the references Dow Corning 2-8179® and Dow Corning 2-8178 Gellant® by the company Dow Corning; the sequential copolymers of “diblock”, “triblock” or “radial” type of the polystyrene/polyisoprene, polystyrene/polybutadiene type, such as those sold under 1 M 5960®).

As solid particles of film-forming polymer according to the invention, use may be made more preferentially of:
aqueous dispersions of acrylic polymer sold under the names Acronal DS-6250® by the company BASF, Neocryl A-45®, Neocryl XK-90®, Neocryl A-1070®, Neocryl A-1090®, Neocryl BT-62®, Neocryl A-1079® and Neocryl A-523® by the company DSM, Joncryl 95® and Joncryl 8211® by the company BASF, Daitosol 5000 AD® (INCI name: Acrylates Copolymer) or Daitosol 5000 SJ®, Epitex 66 Polymer® in emulsion form by the company Dow Corning (INCI name: Acrylates/Ethylhexyl Acrylate Copolymer) by the company Daito Kasey Kogyo, Acudyne 5600P® and Acudyne 5800P® by the company Dow Chemical (INCI name: Acrylates Copolymer);
the aqueous dispersions of polyurethane which are sold under the names

Neorez R-981® and Neorez R-974® by the company DSM, Avalure UR-405®, Avalure UR-410®, Avalure UR-425®, Avalure UR-450®, Sancure 875♦, Avalure UR 445♦ or Avalure UR 450♦ by the company Noveon, Impranil 85♦ by the company Bayer, Baycusan C 1010® (INCI name: Polyurethane-35), such as the product sold under the name Baycusan C 1010/1®, and Baycusan C 1008/1® (INCI name: Polyurethane-48 (and) Methylpropanediol (and) Caprylyl Glycol (and) Phenylpropanol), which are sold by the company Covestro.
sulfopolyesters, such as those sold under the brand name Eastman AQ♦ by the company Eastman Chemical Products,
vinyl dispersions such as Mexomer PAM®, aqueous dispersions of polyvinyl acetate such as Vinybran® from the company Nisshin Chemical or the products sold by the company Union Carbide, aqueous dispersions of vinylpyrrolidone, dimethylaminopropylmethacrylamideand lauryldimethylpropylmethacrylamidoammonium chloride terpolymer such as Styleze W® from ISP,
aqueous dispersions of polyurethane/polyacrylic hybrid polymer such as the products sold under the references Hybridur® by the company Air Products or Duromer® from National Starch,
mixtures thereof.

According to a preferred embodiment, a composition in accordance with the invention comprises an aqueous dispersion of acrylic film-forming polymer.

According to a particular form, a composition in accordance with the invention comprises an aqueous dispersion of solid particles of acrylic film-forming polymer having the INCI name: Acrylates Copolymer, such as the commercial product sold under the name Epitex 66 Polymer® in emulsion form by the company Dow Corning.

### Aqueous Phase

A composition according to the invention comprises an aqueous phase (6).

The term “aqueous phase” means a phase comprising water and also optionally all the solvents and ingredients that are water-soluble or water-miscible (water miscibility greater than 50% by weight at 25° C.), for instance lower monoalcohols containing from 1 to 5 carbon atoms such as ethanol or isopropanol.

The aqueous phase may contain a demineralized water or alternatively a floral water such as cornflower water and/or a mineral water such as Vittel water, Lucas water or La Roche Posay water and/or a spring water.

In particular, a composition according to the invention comprises at least 30.0% by weight, better still at least 40.0% by weight, or even a content of from 50% to 60% by weight of water, relative to the total weight of the composition.

### Fillers (8)

A composition according to the invention comprises at least one filler (8) having a water absorption capacity <200 mL/100 g measured at 25° C. according to the Wet Point method.

The Wet Point is defined as the volume of water in mL per 100 g of filler corresponding to the production of a homogeneous paste when solvent water is added to a powder.

The protocol applied is as follows, inspired by the standard ISO787-5 and suitable for reducing the amount of sample and for fillers only (particles >1 μm).

The principle of the method is to manually prepare powder/water mixtures in variable proportions so as to determine the threshold concentration above which an excess of liquid is perceptible on a macroscopic scale on the basis of naked-eye observations. Below this threshold, the mixture has a granular appearance. This liquid concentration limit is considered as the Wet Point, i.e. the maximum absorption capacity of the powder for this liquid.

The technique consists in weighing out a defined amount of powder, adding a defined amount of liquid, performing a homogenization step using a spatula, then stirring by vortexing and observing the mixture. The object is to proceed iteratively (adding liquid or increasing the liquid/powder ratio) to obtain a pasty, non-granular but non-fluid mixture.
1. Using a precision balance, the filler sample is accurately weighed into a 25 ml scintillation bottle.
2. The same mass of liquid (water) is added precisely.
3. The mixture is homogenized by vortexing for 30 seconds.
4. The mixture is homogenized as a slurry by hand, using a stainless steel spatula.
5. The mixture is homogenized by vortexing for 30 seconds.
6. The bottle is observed by eye and/or using a binocular magnifying glass:
7a. In the case of excess liquid, restart from step 1 in another bottle, then add less liquid in step 2 and continue the protocol up to step 6.
7b. in the case where the water/filler mixture has a granular appearance, restart from step 1 in another bottle, add more water in step 2 and continue the protocol up to step 6;
7c. in cases where the mixture is pasty, non-granular and non-fluid, the amount of water added, expressed in mL, is taken as the “Wet Point” value.

The fillers (8) according to the invention are colorless or white solid particles of any form, which are in a form that is insoluble and dispersed in the medium of the composition. They may be of lamellar, globular, spherical or fibrous form or of any other form intermediate between these defined forms.

The fillers (8) according to the invention may be chosen from inorganic, organic fillers and mixtures thereof.

Among the inorganic fillers (8) having a water absorption capacity <200 mL/100 g that may be used according to the invention, mention may be made of:
clays such as those having the INCI name Magnesium Aluminum Silicate such as the product sold under the trade name Veegum R® (water absorption capacity: 43 mL/100 g) by the company Vanderbilt; those having the INCI name Moroccan Lava Clay such as the product sold under the trade name Ghassoul Powder® (water absorption capacity: 71 mL/100 g) by the company Greentech; those having the INCI name: Aluminum Calcium Sodium Silicate, such as the product sold under the trade name Beadylbeads® (water absorption capacity: 36 mL/100 g) sold by the company US Cosmetics;
talcs, such as the product sold under the trade name J 68 BC® (water absorption capacity: 59 mL/100 g) sold by the company US Cosmetics; the product sold under the trade name Imercare Pharma 00T® (water absorption capacity: 46 mL/100 g) Imerys;
kaolins, such as the product sold under the trade name Imercare 04K® by the company Imeris (water absorption capacity: 48.5 mL/100 g) or the commercial product Kaolin Supreme L′Oréal Decontam® (water absorption capacity: 106 mL/100 g) by the company Imeris; the product sold under the trade name Beraca Amazonian White Clay® (water absorption capacity: 29 mL/100 g) by the company Beraca Ingr. Naturais (Clariant); the product sold under the trade name FJK-16® (water absorption capacity: 49 mL/100 g) by the company Fuji Fine Chemical (Shanghai);
untreated micas of natural origin, such as the product sold under the trade name Sericite SL® (water absorption capacity: 49 mL/100 g) by the company SL Horie Kako; the commercial product Mearlmica SV® (water absorption capacity: 54 mL/100 g) sold by the company BASF; the commercial product Sumicos Velvet Mica 43037® (water absorption capacity: 64 mL/100 g) sold by the company Sudarshan Chemical;
synthetic micas such as those having the INCI name: Synthetic Fluorphlogopite, such as the commercial product Synafil S 1050® (water absorption capacity: 22 mL/100 g) sold by the company Eckart;
chemically-treated micas such as the product having INCI name: Mica (and) Isostearyl Sebacate (and) Disodium Stearoyl Glutamate (and) Aluminum Hydroxide, such as the commercial product NHS-S-100® (water absorption capacity: 40 mL/100 g) sold by the company Myoshi Kasei;
calcium carbonate such as the commercial product Carbomat® (water absorption capacity: 30 mL/100 g) sold by the company Sensient; the commercial product Omyacare Extra 35-OG® (water absorption capacity: 27 mL/100 g) sold by the company Omya;
boron nitride, such as the commercial product Ronaflair Boroneige SQ-6® (water absorption capacity: 84 mL/100 g) sold by the company Merck; the commercial product Boron Nitride SHP 3® (water absorption capacity: 100 mL/100 g) sold by the company Mizushima Ferroalloy,
bismuth oxychloride having the INCI name: Bismuth Oxychloride, such as the commercial product Pearl 2600 UVS® (water absorption capacity: 38 mL/100 g) sold by the company FQ Specialty Chemicals;
glass beads having the INCI name: Glass Beads, such as the commercial product P2015SL® (water absorption capacity: 35 mL/100 g) sold by the company Prizmalite;
uncoated or coated spherical silica powders, such as the commercial product Spherica L-1500® (water absorption capacity: 137 mL/100 g) sold by the company JGC Catalysts & Chemicals; the commercial product Sunsphere H51® or Solesphere H51® (water absorption capacity: 100 mL/100 g) by the company AGC SI-Tech; the commercial product BA4® (water absorption capacity: 29 mL/100 g) sold by the company JGC Catalysts & Chemicals; lauroyl lysine-coated silica powders having the INCI name: Silica (and) Lauroyl Lysine, such as the commercial product Amilon® (water absorption capacity: 123 ml/100 g) sold by the company Ikeda.

Among the organic fillers (8) having a water absorption capacity <200 mL/100 g that may be used according to the invention, mention may be made of:
native starches such as the corn starch having INCI name: Zea Mays Starch, such as the product sold under the trade name Beauty By Roquette ST005® (water absorption capacity: 60 mL/100 g) by the company Roquette; rice starch having the INCI name: Oryza Sativa Starch such as the commercial product Remytec F-I® (water absorption capacity: 69 mL/100 g) sold by the company Creachem;
chemically modified starches, such as potato starch modified with sodium ethylaminodipropionate, having the INCI name: Potato Starch Modified, such as the commercial product Structure Solanace sold by the company AkzoNobel; corn starch esterified with the aluminum salt of octenylsuccinic anhydride, having the INCI name: Aluminum Starch Octenylsuccinate, such as the commercial product Dry Flo Plus® (water absorption capacity: 74 mL/100 g) sold by the company sold by the company AkzoNobel; hydroxypropylated corn starch phosphate having the INCI name: Hydroxypropyl Starch Phosphate Octenylsuccinate, such as the commercial product Structure Zea® (water absorption capacity: 64 mL/100 g) sold by the company AkzoNobel; crosslinked potato sodium carboxymethyl starch having the INCI name: Sodium Carboxymethyl Starch, such as the commercial product Beauty By Roquette ST118® (water absorption capacity: 181 ml/100 g) by the company Roquette; acetylated and oxidized tapioca starch having the INCI name: Oxidized Starch Acetate, such as the commercial product GF-A390® (water absorption capacity: 55 mL/100 g) sold by the company Foshan Gaofeng Starch Technology; composite powders of starch and sodium polyacrylate having the INCI name: Sodium Polyacrylate Starch, such as the commercial products Makimousse 25® (water absorption capacity: 86 mL/100 g) and Makimousse 12® (water absorption capacity: 179 mL/100 g) sold by the company Daito Kasei Kogyo;
silicone elastomer powders such as those having the INCI name:

Dimethicone/Vinyl Dimethicone Crosspolymer, such as the commercial products Dowsil Trefil E-506 S Silicone Powder® (water absorption capacity: 80 mL/100 g) and Dowsil 9701 Cosmetic Powder® (water absorption capacity: 107 mL/100 g sold by the company DOW CORNING; those having the INCI name: Diphenyl Dimethicone/Vinyl Diphenyl Dimethicone/Silsesquioxane Crosspolymer, such as the commercial product KSP 300® (water absorption capacity: 130 mL/100 g) sold by the company Shin-Etsu;
cellulose powders such as the crystalline cellulose powders having the INCI name:

Microcrystalline Cellulose, such as the commercial product Avicel PC 105® (water absorption capacity: 159 mL/100 g) sold by the company DuPont; the cellulose powders having the INCI name: Cellulose such as the commercial product Cellulobeads D-10® (water absorption capacity: 124 mL/100 g) sold by the company Daito Kasei Kogyo;
acrylic powders such as the product having the INCI name: Methyl Methacrylate Crosspolymer (and) Paraffinum Liquidum, such as the commercial product Sepimat H 10® (water absorption capacity: 82 mL/100 g) sold by the company SEPPIC; those having the INCI name: Methyl Methacrylate Crosspolymer, such as the commercial product MSP-930® (water absorption capacity: 63 mL/100 g); those having INCI name: Styrene/Acrylates Copolymer, such as the commercial product Sunspheres Powder® (water absorption capacity: 91 mL/100 g) sold by the company Rohm & Haas;
polyurethane powders such as those having the INCI name: HDI/Trimethylol Hexyl Lactone Crosspolymer, such as the commercial product D-400® (absorption capacity: 138 mL/100 g) sold by the company Toshiki Pigment;
silk protein powders having the INCI name: Silk Powder, such as the commercial product Crosilk Powder-PW® (absorption capacity: 70 mL/100 g) sold by the company Croda.

More particularly, fillers chosen from kaolins, spherical silica powders, crosslinked potato sodium carboxymethyl starch having the following INCI name: Sodium Carboxymethyl Starch, and mixtures thereof, are used.

The filler(s) (8) having a water absorption capacity <200 mL/100 g are preferably present in contents ranging from 0.1% to 10% by weight, preferably from 0.2% to 5% and more preferentially from 0.5% to 3% by weight relative to the total weight of the composition.

### Water-Soluble Liquid Polyol

The composition according to the invention may also comprise at least one water-soluble liquid polyol.

The term “water-soluble liquid polyol” means any organic molecule comprising at least two hydroxyl groups (OH), which is liquid at room temperature (25° C.) and atmospheric pressure and water-miscible (water miscibility of greater than 50% by weight at 25° C. and atmospheric pressure).

Among the water-soluble polyols, mention may be made of compounds constituted of a linear, branched or cyclic, saturated or unsaturated alkyl chain comprising at least two OH functions. The alkyl chain preferably comprises from 2 to 32 carbon atoms, in particular from 3 to 16, more particularly from 3 to 8 carbon atoms, even more particularly from 3 to 8 carbon atoms.

Preferably, the water-soluble liquid polyol may be chosen from glycols such as ethylene glycol, pentaerythritol, trimethylolpropane, propylene glycol, 1,3-propanediol, 1,3-butylene glycol, isoprene glycol, pentylene glycol, hexylene glycol, dipropylene glycol and glycerol (glycerine); polyglycerols such as glycerol oligomers, for example diglycerol; polyethylene glycols, and mixtures thereof.

According to a preferential form of the invention, the water-soluble liquid polyol is 1,3-butylene glycol having the INCI name: Butylene Glycol.

Preferentially, the water-soluble liquid polyol(s) are present in the composition in contents ranging from 0.1% to 10% by weight, preferably from 0.5% to 6% by weight and more preferentially from 1% to 4% by weight relative to the total weight of the composition.

In a preferred embodiment of the invention, the composition according to the invention includes:
at least behenyl behenate, and
at least one mixture of monocetyl phosphate potassium salt, having the INCI name Potassium cetyl phosphate and stearic acid neutralized with aminomethylpropanediol, and
at least one copolymer of vinylpyrrolidone and eicosene, and
at least one semicrystalline polymer chosen from saturated C10 to C30 alkyl (meth)acrylates, and more particularly from poly(stearyl acrylate), poly(behenyl acrylate) and mixtures thereof; and
an aqueous phase, and
solid particles of acrylic film-forming polymer suspended in the aqueous phase (6); and
a filler having a water absorption capacity <200 mL/100 g measured at 25° C. according to the Wet Point method, chosen from kaolins, spherical silica powders, crosslinked potato sodium carboxymethyl starch having the INCI name: Sodium Carboxymethyl Starch, and mixtures thereof; and
1,3-butylene glycol.

### OTHER COMPONENTS

In addition to the abovementioned compounds, a composition according to the invention may, needless to say, comprise additional ingredients.

### a) Fatty Alcohol

Preferentially, the compositions according to the invention also comprise at least one fatty alcohol. A composition may thus comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) are in particular chosen from linear or branched, saturated or unsaturated C14-C30, preferably C14-C24, and better still C14-C20, fatty alcohols.

The fatty alcohol(s) are in particular chosen from linear and saturated C14-C30 fatty alcohols, preferably linear and saturated C14-C24 fatty alcohols, and better still linear and saturated C14-C20 fatty alcohols.

According to a particularly preferred embodiment, the fatty alcohol is in the form of a mixture of several different fatty alcohols, and is preferably a mixture of several linear and saturated C14-C30, better still C14-C24, even better still C14-C20, fatty alcohols.

Preferably, the fatty alcohol according to the invention is chosen from cetyl alcohol (C16), stearyl alcohol (C18) and mixtures thereof (also known as “cetearyl alcohol”). Preferentially, the fatty alcohol according to the invention is a mixture of cetyl alcohol and stearyl alcohol. Such a mixture is notably sold under the name Lannette O OR/MB® by the company BASF.

According to a preferred embodiment, the fatty alcohol is solid at room temperature.

The fatty alcohol is present in the compositions of the invention in amounts ranging from 1.0% to 20.0% by weight, relative to the total weight of the composition, preferably from 2.0% to 15.0% by weight and even more particularly from 3.0% to 10.0% by weight, relative to the total weight of the composition.

### b) Waxes

Thus, a composition according to the invention may also comprise a wax.

However, with regard to the objectives targeted by the present invention, the compositions according to the invention preferably comprise a reduced amount of wax and notably less than 5% by weight, or even less than 3% by weight of waxes, relative to the total weight of the composition.

As specified in the preamble, for the purposes of the invention, the term “waxes” refers to lipophilic compounds, which are solid at room temperature (25° C.) and at atmospheric pressure (760 mmHg), with a reversible solid/liquid change of state, which have a melting point of greater than or equal to 40° C., which may be up to 120° C.

It is recalled that, for the purposes of the invention, the waxes to which the abovementioned amount limitation relates are distinct from those that may be embodied by the component which is a linear fatty acid monoester (1) according to the invention and additional fatty alcohol components as defined below.

This limitation relates more particularly to waxes made up of complex mixtures and which are notably described in Ullmann's Encyclopedia of Industrial Chemistry 2015, Wiley-VCH Verlag GmbH & Co. KGaA.

Such waxes may notably be natural, but may also be synthetic.

The term “natural” wax is intended to denote any wax which pre-exists in nature or which can be converted, extracted or purified from natural compounds which exist in nature.

Among natural waxes, mention may notably be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semirefined waxes, filtered waxes, refined waxes—and microcrystalline waxes, termed microwaxes, including bright stock gatsch. The fossil waxes also contain lignite, also referred to as montan wax, or peat wax.

As natural waxes other than fossil waxes, mention may be made of animal and plant waxes.

As examples of plant waxes, mention may be made of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, mixtures thereof and derivatives thereof.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is approximately 70% composed of esters which are mainly monoesters (of fatty acid and of fatty alcohol), but also of hydroxy esters, of diesters and triesters and esters of sterols, and also of long-chain linear hydrocarbons, of free acids and of free alcohols. For obvious reasons, the weight proportion of their ingredients and their degree of purity are difficult to guarantee from one production batch to another.

The term “synthetic” wax is intended to denote waxes whose synthesis requires one or more chemical reactions performed by a human being.

Among the synthetic waxes, semisynthetic waxes and totally synthetic waxes can be distinguished. Synthetic waxes may be waxes obtained via a Fischer-Tropsch process, for example constituted of paraffins with a number of carbon atoms ranging from 20 to 50, or polyolefin waxes, for example homo- or copolymers of ethylene, of propene or of butene, or even of longer-chain α-olefins. The latter can be obtained by thermomechanical degradation of plastic polyethylene, by the Ziegler process, by high-pressure processes, or also via processes catalyzed by metallocene species. These waxes may be crystallizable, partially crystallizable or amorphous. The abovementioned synthetic waxes are generally nonpolar and can be chemically treated in order to obtain polar waxes, for example by one or more of the following reactions: air oxidation, grafting, esterification, neutralization with metal soaps, amidation, direct copolymerizations or addition reactions.

In this case also, their composition may be constituted of a mixture of ingredients since the fatty-chain lengths are not well defined, thus forming a mixture of compounds having different fatty-chain lengths and for which it is difficult for manufacturers to guarantee perfect reproducibility from one production batch to another.

Consequently, the compositions according to the invention advantageously comprise less than 5% by weight, preferably less than 3% by weight of waxes, notably of multicomponent natural or synthetic wax, relative to the total weight of the composition.

For the purposes of the invention, a multicomponent wax denotes a wax consisting of a mixture of several ingredients, either such as exists naturally like natural waxes, or such as is formed during the process of industrial synthesis of these materials.

In a particularly preferred embodiment of the invention, the composition is free of these waxes, notably multicomponent natural or synthetic wax.

As specified above, the preferred texturing compounds according to the invention are by contrast and advantageously synthetic, single-component compounds, which are thus available in a form purified to more than 99%, like compound (1) required according to the invention.

### c) Liquid Fatty Phase

A composition according to the invention may also comprise a liquid fatty phase.

Such a liquid fatty phase is an organic phase that is liquid at room temperature (25° C.) and at atmospheric pressure (760 mmHg), nonaqueous and water-immiscible.

The liquid fatty phase may contain a nonvolatile oil notably chosen from nonvolatile hydrocarbon-based oils.

The term “hydrocarbon-based oil” refers to an oil mainly containing carbon and hydrogen atoms and possibly one or more functions chosen from hydroxyl, ester, ether and carboxylic functions.

The term “nonvolatile oil” means an oil that remains on the skin or the keratin fiber at room temperature and atmospheric pressure for at least several hours, and that notably has a vapor pressure of less than 10-3 mmHg (0.13 Pa).

A composition according to the invention preferably comprises less than 5.0% by weight and more preferentially less than 2.0% by weight of volatile oil(s), relative to the total weight of the composition. In a particularly preferred embodiment of the invention, the composition is free of volatile oils.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapor pressure, at room temperature and atmospheric pressure, notably having a vapor pressure ranging from 2.66 Pa to 40 000 Pa, in particular ranging from 2.66 Pa to 13 000 Pa and more particularly ranging from 2.66 Pa to 1300 Pa.

### d) Hydrophilic Film-Forming Polymer(s)

A composition according to the invention may preferably comprise at least one hydrophilic film-forming polymer.

For the purposes of the present invention, the term “hydrophilic polymer” means a water-soluble polymer.

For the purposes of the present invention, the term “water-soluble polymer” refers to a polymer which, when introduced into water at a concentration equal to 1%, gives a macroscopically homogeneous solution whose light transmittance, at a wavelength equal to 500 nm, through a sample 1 cm thick, is at least 10%.

For the purposes of the present invention, the term “film-forming polymer” refers to a polymer that is capable, by itself or in the presence of an auxiliary film-forming agent, of forming a macroscopically continuous deposit, and preferably a cohesive deposit, and even better still a deposit whose cohesion and mechanical properties are such that said deposit is isolable and can be manipulated individually, for example when said deposit is prepared by pouring onto a nonstick surface such as a Teflon-coated or silicone-coated surface.

For the purposes of the invention, a hydrophilic film-forming polymer that is particularly advantageous is a (poly)vinylpyrrolidone hydrophilic polymer.

A (poly)vinylpyrrolidone hydrophilic polymer that is suitable for use in the invention may have a weight-average molecular mass, Mw, ranging from 1500 to 500 000 g/mol.

A composition according to the invention has a total solids content of (poly)vinylpyrrolidone hydrophilic polymer(s) of greater than or equal to 0.5% by weight, preferably greater than or equal to 1.0% by weight, more preferentially greater than or equal to 1.5% by weight, relative to the total weight of the composition.

The composition according to the invention preferably comprises from 0.1% to 15.0% by weight, preferably from 0.5% to 10.0% by weight, better still from 1.0% to 8.0% by weight of hydrophilic film-forming polymer(s), relative to the total weight of the composition.

Preferably, the (poly)vinylpyrrolidone hydrophilic polymer(s) in accordance with the invention are linear.

In particular, the (poly)vinylpyrrolidone hydrophilic polymer(s) in accordance with the invention are chosen from random polymers, block copolymers, and a mixture thereof. The term “block copolymer” means a polymer comprising at least two different blocks or sequences and preferably at least three different blocks.

The (poly)vinylpyrrolidone hydrophilic polymer(s) are chosen from:
(poly)vinylpyrrolidone homopolymers;
copolymers of (poly)vinylpyrrolidone/(poly)vinyl acetate, esters,
copolymers of (poly)vinylpyrrolidone/(meth)acrylic, salts thereof, thereof and a mixture thereof.

As (poly)vinylpyrrolidone homopolymers, examples that may be mentioned include:
the polyvinylpyrrolidone (2500 g/mol) sold under the trade name Kollidon 17 PF® by the company BASF,
the polyvinylpyrrolidone sold under the trade name Luviskol K 30 Powder® by the company BASF or sold under the trade name PVP K 30L® by the company ISP (Ashland),
the polyvinylpyrrolidone sold under the trade name PVP K 90® by the company ISP (Ashland).

As (poly)vinylpyrrolidone/(poly)vinyl acetate copolymers, examples that may be mentioned include the vinylpyrrolidone/vinyl acetate (60/40) copolymer sold under the trade name Luviskol VA 64 Powder® by the company BASF.

### e) Cosmetic Active Agents

As cosmetic active agents that may be used in the compositions according to the invention, mention may be made notably of antioxidants, preserving agents, fragrances, neutralizers, cosmetic active agents, for instance emollients, vitamins and screening agents, in particular sunscreens, and mixtures thereof.

These additives may be present in the composition in a content ranging from 0.01% to 15.0% relative to the total weight of the composition.

Needless to say, a person skilled in the art will take care to select the optional additional additives and/or the amount thereof such that the advantageous properties of the composition according to the invention are not, or are not substantially, adversely affected by the envisaged addition.

### f) Dyestuff

A composition according to the invention, and notably those intended for makeup, generally comprises at least one dyestuff such as pulverulent materials, liposoluble dyes or water-soluble dyes.

The pulverulent dyestuffs may be chosen from pigments and nacres.

The pigments may be white or colored, mineral and/or organic, and coated or uncoated. Among the mineral pigments that may be mentioned are titanium dioxide, optionally surface-treated, zirconium oxide, zinc oxide or cerium oxide, and also iron oxide, chromium oxide, manganese violet, ultramarine blue, chromium hydrate and ferric blue. Among the organic pigments that may be mentioned are carbon black, pigments of D & C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica notably with ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the composition according to the invention comprises a pulverulent dyestuff, preferably of pigment type, in particular metal oxides, notably titanium dioxides, iron oxides and mixtures thereof; and more preferentially iron oxides, and more particularly black iron oxides (CI 77499).

Preferably, said dyestuff is present in the composition in a content ranging from 2.0% to 25.0% by weight, preferably from 3.0% to 20.0% by weight, more particularly from 4.0% to 15.0% by weight, relative to the total weight of the composition.

### Physical Characteristics

### a) Solids Content

The composition according to the invention advantageously has a solids content of at least 25.0% by weight, and preferentially of at least 30.0% by weight, relative to the total weight of the composition, or even from 30.0% to 60.0% by weight, relative to the total weight of the composition.

For the purposes of the present invention, the term “solids content” denotes the content of nonvolatile matter.

The amount of solids content (abbreviated as SC) of a composition according to the invention is measured using a Halogen Moisture Analyzer HR 73® commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet mass of the sample, corresponding to its initial mass, and the dry mass of the sample, corresponding to its mass after halogen heating, are measured using a precision balance.

The experimental error associated with the measurement is of the order of plus or minus 2%.

The solids content is calculated in the following manner:

$$[ Math . 1 ]$$

$$Solids content ( expressed in wt \% ) = 100 \times \frac{Dry mass}{Wet mass}$$

### Consistency

Preferably, a composition according to the invention has a consistency at 25° C. characterized by a hardness ≥20 g, more preferentially a hardness ranging from 20 to 200 g, even more preferentially from 30 to 150 g.

The mascara consistency is measured according to the following protocol:

The measuring device is a TA-XT-Plus® sold by the company Staples Micro System, equipped with a cell for measuring a force of 5 kg and with a cylindrical spindle 12.7 mm (½ inch) in diameter made of Delrin. The mascara is thermostatically maintained at 20° C. It is then placed in excess in a container 60 mm in diameter and 22 mm deep using a metal spatula. The product is spread out so as to avoid any air pockets but without pummelling it, so as not to destructure it. The container is subsequently leveled off using a spatula so as to have a surface which is as uniform as possible. The container is then covered with a watch glass so as to limit the evaporation of solvents present in the formula for about 10 minutes. The options chosen for this measurement method are as follows:
Test mode: Compression measurement
Trigger force: 2.0 g
Pre-speed: 0.5 mm/see
Test speed: 0.5 mm/see
Temperature: 20° C.±1° C.
Penetration distance: 5 mm

Three successive measurements are taken at points at least 12 mm apart, at least 10 mm from the edge of the container. The container is held in place during the measurement. The value retained is the mean of the maxima obtained at each measurement.

Such a consistency is particularly advantageous since it is the most suitable for the mascara application device.

The composition may be manufactured via the known processes generally used in the cosmetics field.

The composition used according to the invention may be a makeup composition, a makeup base, notably for keratin fibers, or base coat, a composition to be applied onto makeup, also known as topcoat, or else a composition for treating keratin fibers.

More specifically, the composition according to the invention is in the form of an eyelash product such as a mascara, an eyebrow product or an eye contour product such as an eye liner, and more particularly a mascara.

Such compositions are notably prepared according to the general knowledge of a person skilled in the art.

### Packaging and Application Assembly or Kit

The present invention also relates to an assembly, or kit, for packaging and applying a cosmetic composition for coating keratin fibers, comprising:
a packaging device comprising said cosmetic composition for coating keratin fibers as described previously,
an applicator for said composition.

Said applicator may be integrally attached to a handle member forming a cap for said packaging device. In other words, said applicator may be mounted in a removable position on said device between a closed position and an open position of a dispensing aperture of the device for packaging said composition.

An assembly for coating keratin fibers that is suitable for use according to the invention may comprise an applicator designed to apply said cosmetic composition for coating keratin fibers and, where appropriate, a conditioning device suitable for receiving said composition.

### Applicator

The applicator comprises means for smoothing and/or separating keratin fibers, such as the eyelashes or the eyebrows, notably in the form of teeth, bristles, spikes or other reliefs.

The applicator is arranged to apply the composition onto the eyelashes or the eyebrows, and may include, for example, a brush or a comb.

The applicator may also be used for finishing of the makeup, over a region of the eyelashes or eyebrows that is made up or laden with composition.

The brush may comprise a twisted core and bristles held between the turns of the core, or may be made in yet another way.

The comb is, for example, produced from a single part by molding of a plastic.

In certain exemplary embodiments, the application member is mounted at the end of a stem, which stem may be flexible, which may contribute to improving the comfort during application.

### Packaging Device

The packaging device comprises a container for housing the composition for coating keratin fibers. This composition may then be withdrawn from the container by immersing the applicator therein.

This applicator may be firmly attached to a member for closing the container. This closing member may form a member for gripping the applicator. This gripping member may form a cap to be removably mounted on said container by any suitable means, such as by screwing, click-fastening, coupling, etc. Such a container may thus reversibly house said applicator.

This container can be optionally equipped with a wiper suitable for removing a surplus of product taken up by the applicator.

A process for applying the composition according to the invention to the eyelashes or the eyebrows may also include the following steps:
forming a deposit of the cosmetic composition on the eyelashes or the eyebrows,
leaving the deposit on the eyelashes or the eyebrows, it being possible for the deposit to dry.

It should be noted that, according to another embodiment, the applicator may form a product container. In such a case, a container may, for example, be provided in the gripping member and an internal channel can internally connect this gripping member to the application members in relief.

Finally, it should be noted that the packaging and application assembly may be in the form of a kit, it being possible for the applicator and the packaging device to be housed separately in the same packaging article.

The expressions “between . . . and . . . ”, and “ranging from . . . to . . . ” should be understood as meaning limits included, unless otherwise specified.

In the description and the examples, the percentages are weight percentages, unless otherwise indicated. The percentages are thus expressed by weight relative to the total weight of the composition. The ingredients are mixed in the order and under the conditions that are readily determined by a person skilled in the art.

The invention will now be described by means of examples which are presented for purely illustrative purposes and should not be interpreted as examples that limit the invention.

### Mascara Examples

| | Ex. 1 | Ex. 1a | Ex. 1b |
|---|---|---|---|
| Ingredients(INCIname) | accordingtotheinvention | outsidetheinvention | outsidetheinvention |
| CetearylAlcohol | 4.5 | 4.5 | 4.5 |
| (LanetteOOR ®) | | | |
| Behenylbehenate(C44) | 8.3 | 8.3 | 8.3 |
| (fattyacidmonoester(1)) | | | |
| (KesterWaxK-72 ®) | | | |
| VP/eicosenecopolymer(4) | 2.0 | 2.0 | 2.0 |
| (AntaronV220F ®) | | | |
| PolyC10-30AlkylAcrylate | 3.0 | 3.0 | 3.0 |
| (TegoSP13-6 ®) | | | |
| (semicrystallinepolymer(5)) | | | |
| Stearicacid | 7.7 | 7.7 | 7.7 |
| (fattyacid(2)) | | | |
| (StearineTP1200Pastilles ®)(DUB50P ®)) | | | |
| Ironoxides/CI77499 | 10.0 | 10.0 | 10.0 |
| (SunpuroBlackIronOxideC33- | | | |
| 7001 ®fromSun) | | | |
| PVP | 2.0 | 2.0 | 2.0 |
| (PVPK30L ®) | | | |
| Acrylatescopolymer | 12.0 | 12.0 | 12.0 |
| (aqueoussuspensionoffilm- | | | |
| formingpolymerparticles(6)) | | | |
| (Epitex66Polymer) | | | |
| Phenoxyethanol | 0.6 | 0.6 | 0.6 |
| Caprylglycol | 0.6 | 0.6 | 0.6 |
| Aminomethylpropanediol(base(3)) | 1.3 | 1.3 | 1.3 |
| (AMPDUltraPC ®fromAngus(DowCorning)) | | | |
| Kaolin | 1.0 | — | — |
| (Imercare04K ®-Imerys) | | | |
| Butyleneglycol | 3.0 | — | 3.0 |
| Water | qs100 | qs100 | qs100 |

### Example Preparation Protocol

The compositions were prepared in a Maxilab Olsa® jacketed tank as follows (on 3 kg). At room temperature (25° C.), all the ingredients except for the Acrylates Copolymer were introduced into the tank. The mixture was heated to about 90° C. with stirring. Emulsification was performed for 15 minutes under turbine/blade/scraper stirring (speed 3000/30/42 rpm) The juice was then gradually cooled to 60° C. under paddle/scraper stirring (speed=30/42 rpm) at a rate of 2° C./minute. The acrylate copolymer was then added and the mixture cooled to 35° C. under paddle/scraper stirring (speed=30/42 rpm) at a rate of 2° C./min, gradually deaerating the formula by means of vacuum before emptying at 35° C.

### Skin Evaluation Protocol

10 mg of product were applied to the skin and 15 to-and-fro fingertip strokes were applied to a 4 cm strip.

The play time was evaluated by the ease of spreading on the skin, i.e. the glidance and the period of time over which the formula spread. It was evaluated on a scale from 0 to 5, with 0 corresponding to a short play time and 5 corresponding to a long play time.

The black intensity was evaluated on a scale from 0 to 5, with 0 corresponding to low intensity and 5 corresponding to high intensity.

The appearance of grains (flakes) is evaluated on a scale from 0 to 5, with 0 corresponding to the absence of grains and 5 corresponding to a high presence of grains.

It should be noted that a significant difference can be observed when the difference between two scores is greater than or equal to 1.

The test was performed on six people and the score is given by consensus between these different people.

### Bioskin® Evaluation Protocol

5 mg of product were applied to a Bioskin® support (bioskin plate #10 100311) and 15 to-and-fro fingertip strokes were applied to a 4 cm strip.

The black intensity was evaluated on a scale from 0 to 5, with 0 being low intensity and 5 high intensity.

The appearance of grains (flakes) was evaluated on a scale from 0 to 5, with 0 corresponding to an absence of grains and 5 corresponding to a strong presence of grains.

It should be noted that a significant difference can be observed when the difference between two scores is greater than or equal to 1.

The test was performed on six people and the score is given by consensus between these different people.

The results obtained are shown in the table below.

| | Ex. 1 | Ex. 1a | Ex. 1b |
|---|---|---|---|
| Observations | accordingtotheinvention | outsidetheinvention | outsidetheinvention |
| Playtimeonskin | 4 | 4 | 2.5 |
| Grainsonskin | 1 | 5 | 4.5 |
| Appearanceofthedepositontheskin | 4 | 1 | 1 |
| GrainsonBioskin ® | 1 | 4.5 | 4.5 |
| AppearanceofthedepositonBioskin ® | 4 | 2 | 1 |

It was noted that Example 1a outside the invention without butylene glycol showed a less intense black deposit with the presence of numerous grains.

It should be noted that Example 1b outside the invention without butylene glycol but also without kaolin showed an even less intense black deposit with the presence of numerous grains.

It should be noted that in Example 1 according to the invention, the presence of butylene glycol allowed the play time to be improved.

It should be noted that in Example 1 according to the invention, the presence of kaolin afforded a more intense black deposit and far fewer grains.

The combination of butylene glycol and kaolin in Example 1 according to the invention resulted in a deposit with substantially fewer grains, greater black intensity and improved play time.

### Comparative Tests Highlighting Different Filler Types

The following fillers were compared.

| | | WaterabsorptioncapacityinmL/ |
|---|---|---|
| INCI | Tradename | 100 g |
| Kaolin | Imercare04K ® | 48.5 |
| Silica | SunsphereH51 ® | 100 |
| Sodiumcarboxymethylstarch | BeautybyRoquetteST118 ® | 180 |
| Magnesiumcarbonate | ExtraLightMagnesiumCarbonate ® | 202 |
| Perlite | Optimat2550OR ® | 208 |
| Cellulose | CellulobeadsUSF-X ® | 211 |
| Silica | SunsphereH33 ® | 300 |
| Silicasilylate | DowsilVM-2270AerogelFineParticles ® | 1117 |

Examples 2, 2a and 2b according to the invention and Examples 2c, 2e, 2f and 2g outside the invention were performed.

| | Ex. 2 | Ex. 2a | Ex. 2b | Ex. 2c |
|---|---|---|---|---|
| Ingredients(INCIname) | accordingtotheinvention | accordingtotheinvention | accordingtotheinvention | outsidetheinvention |
| Cetearylalcohol | 4.5 | 4.5 | 4.5 | 4.5 |
| (LanetteOOR ®) | | | | |
| Behenylbehenate(C44) | 8.3 | 8.3 | 8.3 | 8.3 |
| (fattyacidmonoester(1)) | | | | |
| (KesterWaxK-72 ®) | | | | |
| VP/eicosenecopolymer(4) | 2.0 | 2.0 | 2.0 | 2.0 |
| (Antaron V220F ®) | | | | |
| PolyC10-30AlkylAcrylate | 3.0 | 3.0 | 3.0 | 3.0 |
| (TegoSP13-6 ®) | | | | |
| (semicrystallinepolymer(5)) | | | | |
| Stearicacid | 7.7 | 7.7 | 7.7 | 7.7 |
| fattyacid(2) | | | | |
| (StearineTP1200Pastilles ®)(Dub50P ®)) | | | | |
| Ironoxides/CI77499 | 10.0 | 10.0 | 10.0 | 10.0 |
| (SunpuroBlackIronOxideC33-7001 ®fromSun) | | | | |
| PVP | 2.0 | 2.0 | 2.0 | 2.0 |
| (PVPK30L ®) | | | | |
| Acrylatescopolymer | 12.0 | 12.0 | 12.0 | 12.0 |
| (aqueoussuspensionof | | | | |
| film-formingpolymer | | | | |
| particles(6)) | | | | |
| (Epitex66Polymer ®) | | | | |
| Phenoxyethanol | 0.6 | 0.6 | 0.6 | 0.6 |
| Caprylglycol | 0.6 | 0.6 | 0.6 | 0.6 |
| Aminomethylpropanediol(base(3)) | 1.3 | 1.3 | 1.3 | 1.3 |
| (AMPDUltraPC ®fromAngus(DowCorning)) | | | | |
| Kaolin | 1.0 | — | — | — |
| (Imercare04K ®) | | | | |
| Butyleneglycol | 3.0 | 3.0 | 3.0 | 3.0 |
| Silica | — | 1.0 | — | — |
| (SunsphereH51 ®) | | | | |
| Sodiumcarboxymethylstarch | — | — | 1.0 | — |
| (BeautybyRoquetteST118 ®) | | | | |
| Magnesiumcarbonate | — | — | — | 1.0 |
| (ExtraLightMagnesiumCarbonate ®) | | | | |
| Perlite | — | — | — | — |
| (Optimat2550OR ®) | | | | |
| Cellulose | — | — | — | — |
| (CellulobeadsUSF-X) | | | | |
| Silica | — | — | — | — |
| (SunsphereH33 ®) | | | | |
| Silicasilylate | — | — | — | — |
| (DowsilVM-2270AerogelFineParticles ®) | | | | |
| Water | qs100 | qs100 | qs100 | qs100 |

| | Ex. 2d | Ex. 2e | Ex. 2f | Ex. 2g |
|---|---|---|---|---|
| Ingredients(INCIname) | outsidetheinvention | outsidetheinvention | outsidetheinvention | outsidetheinvention |
| Cetearylalcohol | 4.5 | 4.5 | 4.5 | 4.5 |
| (LanetteOOR ®) | | | | |
| Behenylbehenate(C44) | 8.3 | 8.3 | 8.3 | 8.3 |
| (fattyacidmonoester(1)) | | | | |
| (KesterWaxK-72 ®) | | | | |
| VP/eicosenecopolymer(4) | 2.0 | 2.0 | 2.0 | 2.0 |
| (Antaron V220F ®) | | | | |
| PolyC10-30AlkylAcrylate | 3.0 | 3.0 | 3.0 | 3.0 |
| (TegoSP13-6 ®) | | | | |
| (semicrystallinepolymer(5)) | | | | |
| Stearicacid | 7.7 | 7.7 | 7.7 | 7.7 |
| fattyacid(2) | | | | |
| (StearineTP1200Pastilles ®)(Dub50P ®)) | | | | |
| Ironoxides/CI77499 | 10.0 | 10.0 | 10.0 | 10.0 |
| (SunpuroBlackIronOxideC33-7001 ®fromSun) | | | | |
| PVP | 2.0 | 2.0 | 2.0 | 2.0 |
| (PVPK30L ®) | | | | |
| Acrylatescopolymer | 12.0 | 12.0 | 12.0 | 12.0 |
| (aqueoussuspensionof | | | | |
| film-formingpolymer | | | | |
| particles(6)) | | | | |
| (Epitex66Polymer ®) | | | | |
| Phenoxyethanol | 0.6 | 0.6 | 0.6 | 0.6 |
| Caprylglycol | 0.6 | 0.6 | 0.6 | 0.6 |
| Aminomethylpropanediol(base(3)) | 1.3 | 1.3 | 1.3 | 1.3 |
| (AMPDUltraPC ®fromAngus(DowCorning)) | | | | |
| Kaolin | — | — | — | — |
| (Imercare04K ®) | | | | |
| Butyleneglycol | 3.0 | 3.0 | 3.0 | 3.0 |
| Silica | — | — | — | — |
| (SunsphereH51 ®) | | | | |
| Sodiumcarboxymethylstarch | — | — | — | — |
| (BeautybyRoquetteST118 ®) | | | | |
| Magnesiumcarbonate | — | — | — | — |
| (ExtraLightMagnesiumCarbonate ®) | | | | |
| Perlite | 1.0 | — | — | — |
| (Optimat2550OR ®) | | | | |
| Cellulose | — | 1.0 | — | — |
| (CellulobeadsUSF-X) | | | | |
| Silica | — | — | 1.0 | — |
| (SunsphereH33 ®) | | | | |
| Silicasilylate | — | — | — | 1.0 |
| (DowsilVM-2270AerogelFineParticles ®) | | | | |
| Water | qs100 | qs100 | qs100 | qs100 |

### Preparation Protocol

A filler-free preparation was prepared in a Maxilab Olsa® jacketed tank as follows (on 3 kg): At room temperature (25° C.), all the ingredients except for the Acrylates Copolymer and the filler were introduced into the tank. The mixture was heated to about 90° C. with stirring. Emulsification was performed for 15 minutes under turbine/blade/scraper stirring (speed 3000/30/42 rpm) The juice was then gradually cooled to 60° C. under paddle/scraper stirring (speed=30/42 rpm) at a rate of 2° C./minute. The acrylate copolymer was then added and the mixture cooled to 35° C. under paddle/scraper stirring (speed=30/42 rpm) at a rate of 2° C./min, while gradually deaerating the formula by means of vacuum before emptying at 35° C. 1% of each selected filler was added separately to this preparation, which was then homogenized using a Speed-Mixer® for 1 min 30 sec at 2500 rpm.

### Evaluation of Examples 2, 2a and 2b According to the Invention and of Examples 2c, 2d, 2e, 2f and 2g Outside the Invention

| | Ex. 2 | Ex. 2a | Ex. 2b | Ex. 2c |
|---|---|---|---|---|
| Observations | accordingtotheinvention | accordingtotheinvention | accordingtotheinvention | outsidetheinvention |
| Grainsonskin | 1.5 | 1.5 | 1.5 | 4 |
| Appearanceofthedepositontheskin | 4 | 4 | 4 | 2.5 |
| Grainson | 1 | 1 | 1 | 2.5 |
| Bioskin ® | | | | |
| AppearanceofthedepositonBioskin ® | 4 | 3.5 | 4 | 1.5 |

| | Ex. 2d | Ex. 2e | Ex. 2f | Ex. 2g |
|---|---|---|---|---|
| Observations | outsidetheinvention | outsidetheinvention | outsidetheinvention | outsidetheinvention |
| Grainsonskin | 4.5 | 4 | 4.5 | 5 |
| Appearanceofthedepositontheskin | 1.5 | 2 | 1.5 | 1 |
| Grainson | 2.5 | 2 | 3.5 | 4.5 |
| Bioskin ® | | | | |
| AppearanceofthedepositonBioskin ® | 2.5 | 2 | 2 | 2 |

It was noted that Examples 2, 2a and 2b according to the invention incorporating a filler with a water absorption capacity <200 mL/100 g produced a more intense black deposit and fewer grains than Examples 2c, 2d, 2e, 2f and 2g outside the invention comprising a filler with a water absorption capacity >200 mL/100 g.

1. A composition comprising, notably in a physiologically acceptable medium:
(1) at least one linear fatty acid monoester of formula (I) below:
R₁—O—R₂ (I)
in which R₁ and R₂ are linear and saturated and have, independently of each other, a number of carbon atoms greater than or equal to 20, with R₁ representing an acyl radical, and R₂ representing an alkyl radical, and
(2) at least one anionic surfactant, and
(3) at least one base that is capable of partially or totally neutralizing said anionic surfactant, and
(4) at least one vinylpyrrolidone/eicosene copolymer, and
(5) at least one semicrystalline polymer, and
(6) an aqueous phase, and
(7) at least one film-forming polymer in the form of solid particles in suspension in the aqueous phase, and
(8) at least one filler having a water absorption capacity <200 mL/100 g measured at 25° C. by the Wet Point method; and
(9) at least one water-soluble liquid polyol.

2. The composition as claimed in claim 1, wherein if the composition further comprises at least one wax, the composition contains less than 5% by weight of wax(es) relative to the total weight of said composition.

3. The composition as claimed in claim 1, in which the at least one linear fatty acid monoester (1) is chosen from arachidyl arachidate, behenyl behenate, and mixtures thereof.

4. The composition as claimed in claim 1, comprising at least 5.0% by weight of the linear fatty acid monoester(s) (1) relative to the total weight of the composition.

5. The composition as claimed in claim 1, in which the at least one anionic surfactant is present in a content of from 3.5% to 20.0% by weight relative to the total weight of the composition.

6. The composition as claimed in claim 1, in which said anionic surfactant(s) (2) are chosen from:
C12-C20 monoalkyl phosphates;
fatty acids containing from 14 to less than 20 carbon atoms; and
mixtures thereof.

7. The composition as claimed in claim 1, in which said anionic surfactant(s) (2) comprises Stearic acid neutralized with aminomethylpropanediol.

8. The composition as claimed in claim 1, in which said anionic surfactant(s) (2) comprises at least monocetyl phosphate anionic surfactant in its form totally neutralized with potassium hydroxide (KOH).

9. The composition as claimed in claim 6, comprising a mixture of monocetyl phosphate potassium salt and stearic acid neutralized with aminomethylpropanediol.

10. The composition as claimed in claim 1, comprising at least one copolymer of vinylpyrrolidone and eicosene (4), in a concentration from 0.5% to 10% by weight relative to the total weight of the composition.

11. The composition as claimed in claim 1, comprising at least 2.0% by weight of semicrystalline polymer(s) (5), relative to the total weight of the composition.

12. The composition as claimed in claim 1, in which the semicrystalline polymer(s) (5) are derived from a monomer containing a crystallizable chain chosen from saturated C10 to C30 alkyl (meth)acrylates.

13. The composition as claimed in claim 1, in which said semicrystalline polymer(s) (5) comprises a poly(behenyl acrylate).

14. The composition as claimed in claim 1, in which the film-forming polymer(s) in the form of particles in suspension in water (7) is present in a solids content from 0.5% to 10% by weight, relative to the total weight of the composition.

15. The composition as claimed in claim 1, in which the film-forming polymer(s) in the form of particles in suspension in water (7) is Acrylates Copolymer.

16. The composition as claimed in claim 1, comprising at least 30.0% by weight relative to the total weight of the composition.

17. The composition as claimed in claim 1, in which the filler(s) (8) with a water absorption capacity <200 mL/100 g is chosen from inorganic fillers, organic fillers and mixtures thereof.

18. The composition as claimed in claim 17, in which the filler(s) (8) with a water absorption capacity <200 mL/100 g comprises an inorganic filler chosen from:
clays;
talcs;
kaolin;
untreated micas of natural origin;
synthetic micas;
chemically-treated micas;
calcium carbonate;
boron nitride;
bismuth oxychloride;
glass beads; and
uncoated or coated spherical silica powders.

19. The composition as claimed in claim 17, in which the filler(s) (8) with a water absorption capacity <200 mL/100 g comprises an organic filler chosen from:
native starches;
chemically modified starches;
silicone elastomer;
cellulose powders;
acrylic powders;
polyurethane powders; and
silk protein powders.

20. The composition as claimed in claim 1, in which the filler(s) (8) with a water absorption capacity <200 mL/100 g is chosen from kaolins, spherical silica powders, Sodium Carboxymethyl Starch, and mixtures thereof.

21. The composition as claimed in claim 1, in which the filler(s) (8) with a water absorption capacity <200 mL/100 g are present in a content from 0.1% to 10% by weight relative to the total weight of the composition.

22. The composition as claimed in claim 1, in which the water-soluble liquid polyol(s) (9) is Butylene Glycol.

23. The composition as claimed in claim 1, in which the water-soluble liquid polyol(s) (9) is present in the composition in a content from 0.1% to 10% by weight relative to the total weight of the composition.

24. The composition as claimed in claim 1, further comprising at least one dyestuff.

25. The composition as claimed in claim 1, further comprising at least one fatty alcohol.

26. The composition as claimed in claim 25, in which the fatty alcohol(s) is present in an amount of from 1.0% to 20.0% by weight relative to the total weight of the composition.

27. The composition as claimed in claim 1, further comprising at least one water-soluble film-forming polymer.

28. The composition as claimed in claim 27, comprising from 0.1% to 15.0% by weight of the at least one water-soluble film-forming polymer relative to the total weight of the composition.

29. The composition as claimed in claim 1, having a solids content of at least 25.0% by weight relative to the total weight of the composition.

30. The composition as claimed in claim 1, having at 25° C. a consistency characterized by a hardness ≥20 g.

31. The composition as claimed in claim 1, in the form of an eyelash product.

32. An assembly, or kit, for packaging and applying a cosmetic composition for coating keratin fibers, comprising:
a packaging device comprising a composition as defined according to claim 1,
an applicator for said composition.

33. A process for coating keratin material comprising applying to said keratin material at least one composition as defined in claim 1 to the kertain material in an amount sufficient to coat the keratin material.
